(19) United States
(12) Patent Application Publication
(10) Pub. No.: US 2024/0117452 A1
(43) Pub. Date: Apr. 11, 2024

(54) SYSTEMS AND METHODS FOR ISOLATING A TARGET FROM A BIOLOGICAL SAMPLE

(71) Applicants: Salus Discovery, LLC, Madison, WI (US); Flambeau Diagnostics, LLC, Monona, WI (US)

(72) Inventors: Jay Warrick, Madison, WI (US); Brianna Mullins, Deforest, WI (US); Patrick McMinn, Madison, WI (US); Dave Beebe, Monona, WI (US); Ryan Shogren, Madison, WI (US); Mitch Geiger, Madison, WI (US); Franklin Zhong, Madison, WI (US); Duane S. Juang, Madison, WI (US); Terry D. Juang, Madison, WI (US)

(21) Appl. No.: 18/270,440

(22) PCT Filed: Jan. 5, 2022
(86) PCT No.: PCT/US2022/011307

§ 371 Date: Jun. 29, 2023

Related U.S. Application Data
(60) Provisional application No. 63/134,464, filed on Jan. 6, 2021.

(51) Int. Cl. C12Q 1/70; B01L 3/00; B01L 3/02; C12Q 1/6844

(52) U.S. Cl. CPC C12Q 1/701; B01L 3/0237; B01L 3/5085; C12Q 1/6844; C12Q 1/6806

(57) ABSTRACT

Provided herein are systems and methods for isolating a target from a biological sample. In some aspects, provided herein are automated systems and methods for isolating a target from a plurality of biological samples. For example, provided herein are automated systems and methods for isolating viral nucleic acid from a biological sample using a magnetic force to draw the nucleic acid out of a liquid biological sample.

### STATEMENT REGARDING RELATED APPLICATIONS

This application claims priority to U.S. Provisional Patent Application No. 63/134,464, filed Jan. 6, 2021, the entire contents of which are incorporated herein by reference for all purposes.

### STATEMENT OF FEDERAL SUPPORT

This invention was made with government support under R43 OD023021-01A1 awarded by the National Institutes of Health. The government has certain rights in the invention.

### FIELD

Provided herein are systems and methods for isolating a target from a biological sample. In some aspects, provided herein are automated systems and methods for isolating a target from a plurality of biological samples. For example, provided herein are automated systems and methods for isolating nucleic acid from a biological sample using a magnetic force to draw the nucleic acid out of a liquid biological sample.

### BACKGROUND

The ability to extract or separate a target analyte (e.g., nucleic acid, protein, whole cell) from a complex background is a critical prerequisite for many common analytical processes in diagnostics, biological research, biomarker discovery, forensics, and more. However, conventional analyte isolation processes are time-consuming, expensive, and laborious, often becoming the bottleneck within the analytical process. Further, some methodologies damage the sample or cause undesired loss or inconsistent yield of sample. Accordingly, improved methods for extraction of a target analyte from a sample are needed.

### SUMMARY

In some aspects, provided herein are methods, systems and devices for separating or isolating one or more targets from a biological sample. The methods described herein generally involve mixing a biological sample with magnetic particles (e.g., paramagnetic particles (PMPs)) to generate a composition comprising one or more target-PMP complexes, for example, and subsequently isolating the target-PMP complexes from the composition. Certain embodiments make use of a device configured to hold a stable fluid level, such as pipettes and pipette tips, which are used to extract, measure, transfer and/or dispense liquid and semi-liquid samples.

In some embodiments, the methods involve generating a liquid/air interface proximal to a bottom opening of a pipette tip, and isolating target-PMP complexes by applying a magnetic force to draw target-PMP complexes through the liquid/air interface. In some embodiments, the methods involve a preconcentration step of drawing target-PMP complexes together to generate a concentration of target-PMP complexes prior to isolating the target-PMP complexes from the composition.

In some embodiments, the methods comprise mixing the biological sample with magnetic particles (e.g., PMPs) to generate a composition comprising one or more target-PMP complexes within a pipette tip, generating a liquid/air interface proximal to a bottom opening of a pipette tip, and applying a magnetic force to the composition to draw target-PMP complexes through the liquid/air interface and into a sample collection device.

In some embodiments, the method comprising performing the following steps, in order: mixing the biological sample with paramagnetic particles (PMPs) to generate a composition comprising one or more target-PMP complexes, aspirating the composition into a pipette tip through a bottom opening of the pipette tip, generating a liquid/air interface proximal to the bottom opening of the pipette tip, and applying a magnetic force the composition to draw some or all of the one or more target-PMP complexes through the liquid/air interface and into a sample collection device. In some embodiments, generating the liquid/air interface proximal to the bottom opening of the pipette tip comprises further aspirating the composition within the pipette tip while the bottom opening of the pipette tip is exposed to air.

In some embodiments, methods for isolating a target from a biological sample comprise performing the following steps, in order: mixing the biological sample with paramagnetic particles (PMPs) to generate a composition comprising one or more target-PMP complexes, aspirating the composition into a pipette tip through a side opening of the pipette tip while a bottom opening of the pipette tip is in conformal contact with a surface such that liquid is unable to enter the pipette tip through the bottom opening, thereby generating a liquid/air interface proximal to the bottom opening of the pipette tip and applying a magnetic force to the composition to draw some or all of the one or more target-PMP complexes through the liquid/air interface and into a sample collection device.

In some embodiments, methods for isolating a target from a biological sample comprise aspirating the biological sample into a pipette tip containing lyophilized paramagnetic particles (PMPs) particles to generate a composition comprising one or more target-PMP complexes within the pipette tip, generating a liquid/air interface proximal to a bottom opening of the pipette tip, and applying a magnetic force to the composition to draw some or all of the one or more target-PMP complexes through the liquid/air interface and into a sample collection device. Generating the liquid/air interface proximal to the bottom opening of the pipette tip may comprise further aspirating the composition within the pipette tip while the bottom opening of the pipette tip is exposed to air.

In some embodiments, methods for isolating a target from a biological sample comprise aspirating the biological sample into a pipette tip containing lyophilized paramagnetic particles (PMPs) to generate a composition comprising one or more target-PMP complexes within the pipette tip. In some embodiments, aspiration occurs through a side opening of the pipette tip while a bottom opening of the pipette tip is in conformal contact with a surface such that liquid is unable to enter the pipette tip through the bottom opening, thereby generating a liquid/air interface is proximal to the bottom opening of the pipette tip. The methods further comprise applying a magnetic force to the composition to draw target-PMP complexes through the liquid/air interface and into a sample collection device.

For any of the methods for isolating a target from a biological sample described herein, the method may comprise a preconcentration step involving applying a magnetic force to the composition to generate a concentration of target-PMP complexes proximal to the liquid/air interface prior to drawing the target-PMP complexes through the liquid/air interface and into the sample collection device.

Any suitable biological sample may be used in the methods described herein. In some embodiments, the biological sample is a nasopharyngeal sample, an oropharyngeal sample, an oral swab sample, an oral sponge sample, a nasal swab sample, a mid-turbinate sample, or a saliva sample. In some embodiments, the biological sample is a saliva sample. The saliva sample may further comprise a reducing agent. For example, the saliva sample may further comprise dithiothreitol (DTT).

In some embodiments, paramagnetic particles are contained in a liquid. The liquid containing the paramagnetic particles may further comprise a reducing agent. For example, the liquid comprising the paramagnetic particles may comprise DTT. In some embodiments, the paramagnetic particles are contained in a lyophilized formulation.

In some embodiments, the sample collection device comprises a wash buffer. In such embodiments, the method may further comprise removing the wash buffer from the sample collection device and adding reagents for detection of the target(s) to the sample collection device after removal of the wash buffer. In some embodiments, reagents for detection of the target comprise reagents for loop-mediated isothermal amplification (LAMP)-based detection of the target.

In some embodiments, the sample collection device comprises reagents for detection of the target(s). The reagents for detection of any target contained within the sample collection device may comprise reagents for loop-mediated isothermal amplification (LAMP)-based detection of the target or targets.

In some embodiments, the sample collection device comprises a multi-well plate. In some embodiments, the sample collection device further comprises a layer of an oil, for example, a mineral oil. For example, the sample collection device may contain a layer of mineral oil that floats above the reagents for detection of the target and/or the wash buffer.

In some embodiments, the biological sample is obtained from a subject suspected of having an infection. For example, the subject may be suspected of having a viral infection. In some embodiments, the subject may be suspected of having a viral upper respiratory infection. In some embodiments, the subject is suspected of having an infection selected from SARS-CoV2, coronavirus, rhinovirus, influenza, respiratory syncytial virus, adenovirus, parainfluenza, human immunodeficiency virus, human papillomavirus, rotavirus, hepatitis C virus, zika virus, Ebola virus, tuberculosis, Borrelia burgdorferi, Staphylococcus, Aspergillus, and Streptococcus. pyogenes. In some embodiments, the target comprises viral nucleic acid.

Any of the methods described herein may be automated. Any of the methods described herein may be performed on a single biological sample or performed simultaneously on a plurality of biological samples. For example, the methods may be performed simultaneously on a plurality of biological samples obtained from distinct individuals. Each biological sample may be aspirated into a separate pipette tip. Biological samples may also be pooled for efficiency and tested first, before identifying a specific sample or samples in the pooled set of test samples that led to a positive result.

The methods described herein may further comprise detecting the target within the sample collection device. In some embodiments, detecting a target within the sample collection device comprises performing a colorimetric, fluorescent, absorbance, or a phosphorescence assay, by way of example. For example, the assay may comprise a colorimetric LAMP, a colorimetric RT-LAMP, a fluorescent LAMP, or a fluorescent RT-LAMP assay. Other nucleic assays may be used, e.g., RT-PCR, as may protein detection assays. Other assays that may be used include, for example, ELISAs, RIAs, which may be quantitative, semi-quantitative, or qualitative. Serology assays to detect IgM, IgG or total antibodies may also be used to evaluate past exposure and immune status, for example.

In some aspects, provided herein are systems for automated isolation of one or more targets from a plurality of biological samples. In some embodiments, the system comprises a plurality of components. In some embodiments, the system comprises a plurality of pipette tips, a multichannel pipette, an apparatus for operating the multichannel pipette, a preconcentration magnet, a sample collection device, a sample collection magnet, and a computer processor configured to operate said multichannel pipette so as to collect a liquid sample in said plurality of pipette tips when attached to said multichannel pipette and to generate an air gap at an open end of said pipette tips following or during collection of said liquid sample in accordance with the methods described and claimed herein. In some embodiments, the system further comprises a moveable surface. One or more of the components may be housed on the moveable surface. The moveable surface may change orientation and/or move in the x-y plane and/or move in the vertical z-direction. In some embodiments, the system further comprises means for storing and displaying results to a user.

In some embodiments, the system further comprises a plurality of biological samples, wherein each of the plurality of biological samples occupies a separate well in a multi-well plate.

In some embodiments, the plurality of pipette tips contain lyophilized paramagnetic particles.

In some embodiments, the system further comprises a mixing container for combining the biological sample with paramagnetic particles (PMPs). In some embodiments, the mixing container comprises a multi-well plate, and each well in the multi-well plate contains PMPs. The PMPs may be lyophilized or contained in a liquid.

In some embodiments, the sample collection device comprises a multi-well plate. The sample collection magnet may be positioned below the sample collection device.

In some embodiments, the apparatus for operating the multichannel pipette is configured to induce movement of the multichannel pipette. In some embodiments, the apparatus for operating the multichannel pipette is configured to aspirate and/or inject liquid when pipette tips are attached to the multichannel pipette.

In some embodiments, the system comprises a moveable surface. The moveable surface may be controlled by a computer.

### BRIEF DESCRIPTION OF THE DRAWINGS

FIG. 1A shows time to threshold values for contrived saliva samples containing 10⁴ viral copies/mL.

FIG. 2A shows time to threshold values for contrived saliva samples obtained from 7 different subjects. DTT was added to the samples before or after heating the samples, and time to threshold values were compared. FIG. 2B shows time to threshold values for virion controls subjected to the same saliva treatment, air purification, and LAMP analysis steps as above.

FIG. 3 shows an exemplary system and a schematic of steps that may be used to isolate PMPs (e.g. target-PMP complexes) from a composition.

FIG. 4 shows another exemplary system and a schematic of steps that may be used to isolate PMPs (e.g. target-PMP complexes) from a composition.

FIG. 5 shows an exemplary embodiment using a tip designed to have a side opening, such that the liquid/air interface can be generated during aspiration of the biological sample.

FIG. 6 shows an exemplary layout of a system that may be used to perform the methods for isolating a target described herein. The system is shown prior to placement of the proper components within the system.

FIG. 7A shows an exemplary layout of a system that may be used to perform the methods for isolating a target described herein (FIG. 7A). The system is shown after placement of the proper components within the system, where 1 is a Waste Chute, 2 is a Mixing Plate, 3 is a Reagent Source Plate, 4 is a Tip Box, 5 is a Sample Rack, 6 and 7 are Tip Boxes, 8 is a Reaction Plate and 9 is a Tip Box (FIG. 7B).

### DEFINITIONS

To facilitate an understanding of the present disclosure, a number of terms and phrases are defined below:

As used herein, “computer system,” “computing device,” “computer,” and analogous expressions refer to one or more devices including at least one tangible computing element. Examples of a tangible computing element include but are not limited to a microprocessor, application specific integrated circuit, programmable gate array, and the like. Examples of a computing device include but are not limited to a mobile computing device such as a smart phone or tablet computer, a wearable computing device (e.g., smart glasses), a laptop computer, a desktop computer, or any other form of computing device. A computing device preferably includes or accesses storage for instructions used to perform steps such as those described herein, access samples or data on which those steps may be performed, and access reagents and materials.

As used herein, “computer-implemented” and analogous expressions refer to technology implemented using, by, with or on one or more computing systems or computer devices.

As used herein, “cause to be displayed,” “causing to be displayed” and analogous expressions refer to taking one or more actions that result in displaying. A computing device, under control of program code, may cause to be displayed an image, picture and/or text for example, and other information to any user of the computing device. For example, a computing device may cause images, pictures, graphs and/or texts—including sample information, patient information, system information, data and test results, etc.—to be displayed directly to a user. For another example, a local or remote server computer under control of program code may cause information to be displayed by making the information available for access by a local computer or mobile device, for example, over a network, such as the Internet, which information the local computer or mobile device may then display to a user of the computer or the mobile device.

As used herein, the terms “detect”, “detecting”, or “detection” may describe either the general act of discovering or discerning or the specific observation of a detectably labeled composition. The term “detecting” when used in reference to a target refers to detecting either the presence or the absence of the target in the sample. In some embodiments, “detecting” a target in a sample refers to determining that the target is present in the sample. In some embodiments, “detecting” a target in a sample refers to determining that the target is not present in the sample or is not present in sufficient quantities to be detected in the sample.

As used herein, the term “biological sample” is used in the broadest sense and is inclusive of many sample types that may be obtained from a subject. Biological samples may be obtained from animals (including humans) and encompass fluids (e.g. urine, blood, blood products, sputum, saliva, etc.), solids, tissues, and gases. Biological samples include saliva, blood products, such as plasma, serum and the like. In some embodiments, the biological sample is a nasopharyngeal sample, an oropharyngeal sample, oral swab or sponge sample, a nasal swab sample, a mid-turbinate sample, or a saliva sample. In some embodiments, the biological sample is a saliva sample. The term “saliva sample” as used herein refers to a sample of saliva collected from a subject. In some embodiments, the biological sample is a nasopharyngeal (NP) sample. A “nasopharyngeal sample” refers to a specimen collected using a swab inserted into the nasopharyngeal cavity of a subject. The biological sample may be subjected to various pre-treatment steps prior to performing a method as described herein. For example, the biological sample may be frozen, heated, mixed with various denaturants (e.g. guanidium thiocyanate), mixed with viscosity reducing reagents (e.g. DTT), mixed with inhibitors of target degradation (e.g. protease inhibitors, RNAse inhibitors, etc.), mixed with various buffers, or subjected to other suitable pre-treatment steps. Any of the substances added to the biological sample (e.g. denaturants, viscosity reducing reagents, inhibitors of target degradation, buffers, etc.) may be added to the biological sample or may be present in a storage buffer present in a container into which the sample is collected (e.g. present within a storage buffer in a sample collection tube). In some embodiments, samples contain or are suspected of containing a microorganism (e.g. a pathogenic or disease-causing microorganism).

The term “subject” as used herein refers to an entity from which a sample is obtained. The subject may be a mammal. In some embodiments, the subject is a human.

The term “target” as used herein is used in the broadest sense and refers to any desired material that may bind a paramagnetic particle and be pulled from a sample by application of a magnetic force. In some embodiments, the target is a protein (e.g. antibody), whole cell, or a nucleic acid (e.g. DNA, RNA). In some embodiments, the target is a metabolite, a carbohydrate, a glycopeptide, or a lipid.

### DETAILED DESCRIPTION

In some aspects, provided herein are systems, methods, and devices including programed or programmable devices, including integrated circuits, central processing units and or computers for separating or isolating one or more targets from a biological sample.

The methods described herein generally involve mixing a biological sample with magnetic particles (e.g., paramagnetic particles (PMPs)) in a device configured to hold a stable fluid level, such as a pipettes and/or a pipette tip to generate a composition comprising one or more target-PMP complexes, for example, and subsequently isolating the target-PMP complexes from the composition.

The methods described herein may be performed for isolation of any desired target from the sample. For example, the target may be a nucleic acid (e.g. DNA, RNA, or various subtypes thereof including mRNA) a protein, a metabolite, a carbohydrate, a glycopeptide, or a lipid. For example, the target may be DNA or RNA. In some embodiments, the target may be nucleic acid or proteins (e.g. antibodies) resulting from a pathogen infecting the subject from which the biological sample was obtained. For example, the target may be bacterial nucleic acid (e.g. bacterial DNA or RNA) or viral nucleic acid (e.g. viral DNA or RNA). As another example, the target may be antibodies produced by the subject in response to infection with the pathogen.

The methods described herein may be performed for isolating a target from any desired biological sample. In some embodiments, the biological sample is a nasopharyngeal sample, an oropharyngeal sample, an oral swab or sponge sample, a nasal swab sample, a mid-turbinate sample, or a saliva sample. In particular embodiments, the biological sample is a saliva sample. In other embodiments, the biological sample is an NP sample.

The biological sample may be collected and/or stored in a suitable container (e.g. a sample collection container) prior to processing the samples by the methods described herein. Any type of sample collection container may be used that is suitable for receiving a sample and storing the sample until performing the described methods for detection of the target. Examples of sample collection containers include, but are not limited to, tubes containing a reversibly removal cap, bags, syringes, droppers, and the like. In some embodiments, the biological samples are pre-treated prior to aspirating into a pipette tip as described herein. For example, the biological samples may be pre-treated in the sample collection container. As another example, the biological samples may be moved to a suitable second container and pre-treated within said second container.

In some embodiments, the biological samples may be pre-treated to inactivate potential pathogens (e.g. virus, bacteria) within the sample. In some embodiments, the biological samples may be pre-treated to lyse cells within the sample, thus releasing the target (e.g. nucleic acid) for subsequent detection. In such embodiments, a pre-treatment step accomplishes both cell lysis (e.g. release of nucleic acid) and inactivation of potential pathogens within the biological sample. In some embodiments, the biological samples may be pre-treated by freezing, heating and/or the addition of a denaturant to the sample. For example, the biological sample may be pre-treated by heating to a sufficient temperature for a suitable duration of time to inactivate potential pathogens within the sample. For example, the biological sample may be heated to about 40° C. or higher. For example, the biological sample may be heated to about 40° C., 45° C., 50° C., 55° C., 60° C., 65° C., 70° C., 75° C., 80° C., 85° C., 90° C., 95° C., 100° C., or more than 100° C. The sample may be maintained at the heated temperature for a suitable duration of time, such as 5 minutes, 10 minutes, 20 minutes, 30 minutes, 40 minutes, 50 minutes, 60 minutes, or more than 1 hour. In particular embodiments, the sample may be heated to 98° C.-100° C. for 5 minutes to accomplish both cell lysis and viral inactivation in a single heat treatment step. In some embodiments, pre-treating the sample comprises adding a denaturant to inactivate potential pathogens within the sample. For example, suitable denaturants include guanidine-based denaturants (e.g. guanidine hydrochloride, guanidine thiocyanate, etc.) and surfactants (e.g., Triton X-100, tween20). In some embodiments, the biological sample does not contain a denaturant. For example, in some embodiments the biological sample (e.g. saliva sample) may not contain a guanidine-based denaturant. In some embodiments, the biological sample (e.g. saliva sample) contains less than 0.3M of a guanidine-based denaturant. For example, the biological sample (e.g. saliva sample) may contain less than 0.3M, less than 0.25M, less than 0.2M, less than 0.15M, less than 0.1M, or less than 0.5M of a guanidine-based denaturant.

The viscosity of certain biological samples (e.g. saliva) makes sample handling difficult. Moreover, the viscosity of samples collected from different individuals varies, introducing potential issues with variability of sample collection between subjects. For example, a saliva sample with high viscosity may result in less volume of saliva successfully being pipetted into a desired container (e.g. for subsequent detection of a pathogen in the sample) compared to saliva with decreased viscosity. This introduces potential downstream issues for inaccurate results, including false negative results. In some embodiments, the biological samples may be pre-treated to reduce viscosity of the sample and thereby improve sample handling in subsequent processing steps. In particular embodiments, the pre-treatment step may be performed to inactivate pathogen(s) within the sample and reduce the viscosity of the sample in one step. In some embodiments, one or more agents to decrease viscosity may be added to the biological sample prior to aspirating the sample into the pipette tip. In some embodiments, the agent to decrease viscosity is a reducing agent. Suitable reducing agents include, for example, dithiothreitol (DTT), tris(2-carboxyethyl)phosphine (TCEP), or 2-mercaptoethanol.

Any suitable amount of a reducing agent may be added to the biological sample (or present in the storage buffer in which the biological sample is placed upon collection). In some embodiments, suitable concentrations of reducing agents may range from 0-500 mM. For example, suitable concentrations of DTT or TCEP may range from 0-250 mM (e.g. 0 mM, about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, or about 250 mM). For example, dithiothreitol (DTT)) may be added to a biological sample (e.g. a saliva sample) at a suitable concentration to decrease viscosity of the sample. In some embodiments, DTT may be added to achieve a 1× concentration within the saliva sample. As another example, suitable concentrations of 2-mercaptoethanol may range from 0-500 mM (e.g. 0 mM, about 25 mM, about 50 mM, about 75 mM, about 100 mM, about 125 mM, about 150 mM, about 175 mM, about 200 mM, about 225 mM, about 250 mM, about 275 mM, 300 mM, about 325 mM, about 350 mM, about 375 mM, about 400 mM, about 425 mM, about 450 mM, about 475 mM, or about 500 mM.

In some embodiments, the viscosity reducing agent (e.g. DTT) is added to the biological sample prior to heating the sample (e.g. to inactivate pathogens and/or induce cell lysis). In some embodiments, the viscosity reducing agent may be present in a sample storage buffer to which the biological sample is added after collection. In some embodiments, the viscosity reducing agent is added to the biological sample after heating the sample. In some embodiments, freezing the sample may be performed to reduce the viscosity of the sample. Any suitable pre-treatment step or combination of pre-treatment steps may be performed to achieve the desired result (e.g. cell lysis, pathogen inactivation, and/or reduction of viscosity of the sample).

The biological sample may additionally comprise a suitable detergent. For example, the biological sample may comprise an ionic detergent (e.g. sodium dodecyl sulfate, deoxycholate, cholate, etc.), a non-ionic detergent (e.g. Triton X-100, DDM, digitonin, Tween 20, Tween 40, Pluronic F-127), a zwitterionic detergent, or a chaotropic detergent. In some embodiments, biological sample comprises 0-5% detergent (v/v). For example, the biological sample may comprise 0%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, or about 5% detergent. The detergent may be added to the biological sample or present in a sample storage buffer to which the biological sample is added upon collection.

In some embodiments, the biological samples comprises a non-ionic detergent (e.g. Triton X-100). For example, the biological sample may comprise 0.001-0.1% Triton X-100. The biological sample may be brought to a suitable volume for subsequent use by the addition of a suitable buffer. For example, the biological sample may be brought to a suitable volume by the addition of phosphate buffered saline (PBS), universal transport medium (UTM), saline, and the like. Such buffers may be added to the biological sample or present in a sample storage buffer to which the biological sample is added upon collection. The biological sample may comprise one or more enzymes or chemical agents to assist with breaking down the contents therein to facilitate release of the desired target. For example, the biological sample may comprise one or more enzymes, such as one or more proteases. In particular embodiments, the biological sample may comprise proteinase K. The biological sample may additionally comprise one or more suitable reagents to prevent degradation of the target within the sample. For example, suitable buffers and/or inhibitors (e.g. RNase inhibitors, nuclease inhibitors, etc.) may be added to the biological sample prior to aspirating the sample into the pipette tip.

In some embodiments, the methods comprise aspirating the biological sample into a pipette tip (or other liquid-holding compartment). In some embodiments, the pipette tip may already contain reagents prior to aspiration of the biological sample. Reagents could be in liquid or dry/lyophilized format (e.g., lyophilized magnetic beads functionalized for a target). In some embodiments, the pipette tip contains lyophilized paramagnetic particles (PMPs).

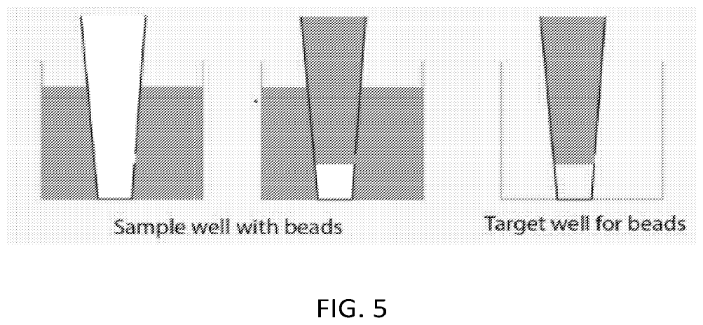

In some embodiments, the biological sample is aspirated through a bottom opening of the pipette tip. In other embodiments, the biological sample is aspirated through an opening on the side of the pipette tip (e.g. a side opening). For example, the pipette tip may contain a bottom opening and a side opening, and the bottom of the pipette tip may be placed against a surface such that the pipette tip and the surface have conformal contact (e.g., the bottom of the pipette tip is flush against the surface) and no liquid is able to enter the pipette tip through the bottom opening. Once the appropriate contact between the bottom opening and the surface is achieved, liquid may be aspirated through the side opening of the pipette tip. Such a method would inherently generate a pocket of air proximal to the bottom opening of the pipette tip during aspiration of liquid through the side opening, as shown in FIG. 5. Pipettes and pipette tips may be disposable and may be single-channel or multichannel, as mentioned. Graduated and repeat dispensing pipettes may also be used.

In some embodiments, the surface of the pipette tip is patterned to encourage or discourage interaction with the reagents. In most cases, this involves patterning of hydrophilicity/hydrophobicity and/or lipophilicity/lipophobicity. Patterning can be achieved via structural patterning (inclusion/exclusion of edges or boundaries and scaling of features to increase or decrease the dominance of surface tension effects), surface texture (use of micropillars on a hydrophobic surface to make it superhydrophobic), or modification of surface chemistry (e.g., chemical or oxygen plasma treatment of polymers). In some embodiments, such patterning methods are used to help stabilize or encourage positioning of the liquid-air boundary used during PMP transfer. Likewise, patterning can be used to encourage capillary filling of reagent to a specified height within the tip (e.g., a closed polypropylene tip dipped into oil to change the surface energy will fill slightly via capillary without aspiration via the pipette mechanism). Patterning can be used to prevent wetting of surfaces as well for certain tip designs. In some embodiments, patterning is used to discourage the sample from sticking to the pipette material in the region of the air-gap. In some embodiments, the air-gap region at the end of the pipette tip is coated with oil via dipping as a means of hydrophobic patterning.

In some embodiments, the tip geometry is altered to facilitate the entrapment of air as a means to facilitate stabilization of the air-liquid boundary during transfer of PMPs from the pipette into/onto the collection devices. In some embodiments, a cylindrical skirt is added to the bottom end of the pipette tip. Thus, concentric annuli are formed where the inner annulus is the original pipette and the outer annulus is the skirt. The top of the outer annulus starts above the bottom of the inner annulus and extends below the bottom of the inner annulus. Therefore, when lowered into a fluid, the outer annulus interacts with fluid first, trapping air between the sample and the inner annulus. When used to aspirate a biological sample, the outer annulus fills first until the inner annulus is reached, then fluid fills the inner annulus trapping a ring of air between the outer and inner annuli. The tip is removed from the sample and additional air is aspirated, moving the fluid initially left in the outer annulus, into the inner annulus and leaving the outer annulus filled with air again and fluid pinned at the bottom opening of the inner annulus. Upon subsequent placement into/onto a collection device that has fluid, air is again trapped in the outer annulus, naturally generating an air-gap between the fluid at the collection device and the biological sample in the inner annulus. Magnetic force can then be applied to transfer the particles. This structure is advantageous because it avoids the need for potentially complicated chemical patterning of phobicity and establishes a robust and reproducible air-gap between the sample and fluid in/on the collection device. Patterning of phobicity/philicity (structural, textural, or surface chemistry) can also be used to make operation of this embodiment even more robust.

Any suitable volume of the biological sample may be aspirated into the pipette tip. For example, about 1 μL to about 500 μL of the biological sample may be aspirated into the pipette tip. In some embodiments, a suitable volume of a saliva sample mixed with DTT (or another agent to reduce viscosity of the saliva) is aspirated into the pipette tip. For example, about 1-500 μL of saliva pre-treated with DTT may be aspirated into the pipette tip. For example, 1 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, or 500 μL of saliva (or saliva mixed with DTT) may be aspirated into the pipette tip.

In some embodiments, the methods comprise mixing the biological sample with capture particles (e.g. magnetic or paramagnetic particles (PMPs). Mixing the biological sample with PMPs allows the PMPs to bind to the target within the sample, thus generating one or more target-PMP complexes. The capture particles (e.g. PMPs) may be contained in a liquid formulation. Alternatively, the capture particles (e.g. PMPs) may be in a lyophilized formulation. For example, lyophilized PMPs may be present in the pipette tip, such that aspirating the biological sample into the pipette tip initiates the process of mixing the biological sample with the PMPs. In some embodiments, the methods comprise mixing the biological sample with PMPs within a pipette tip to generate a plurality of target-PMP complexes within the pipette tip. In some embodiments, the methods comprise mixing the biological sample with a liquid composition comprising capture particles (e.g., paramagnetic particles (PMPs)) to generate a diluted biological sample within the pipette tip.

Any suitable paramagnetic particle may be used. In some embodiments, paramagnetic particles may be purchased from a commercial vendor. The specific type of paramagnetic particle used depends on the target to be isolated from the biological sample. For example, particles with a relatively large surface area may be preferable for binding nucleic acid, such as viral RNA. In some embodiments, the paramagnetic particles may be functionalized to aid in capture/purification of the target. For example, the paramagnetic particles may be functionalized with one or more antibodies, aptamers, or other suitable agents to assist with capture of a target. In some embodiments, the paramagnetic particles may be functionalized with one or more spike protein antibodies to assist with the capture of SARS, coronavirus, SARS-CoV-2 and related targets.

Numerous particles are available and may be selected for use in the methods and systems of the invention, including, for example, those described in “Magbeads 101: A guide to choosing and using magnetic beads,” which notes various useful capture particles that may be modified, coated, blocked or conjugated for specificity or functionality (https://www.cytivalifesciences.com/en/us/news-center/magnetic-beads-a-simple-guide-10001).

Any suitable amount of PMPs may be mixed with the biological sample. In embodiments where the PMPs are contained in a liquid, any suitable volume of the liquid composition comprising paramagnetic particles may be mixed with the biological sample. In some embodiments, the volume of the liquid composition comprising the PMPs may equal or exceed the volume of the biological sample. For example, the volume of the liquid composition comprising PMPs may be at least 100%, at least 150%, at least 200%, at least 250%, at least 300%, at least 350%, at least 400%, at least 450%, or at least 500% the volume of the biological sample.

Any suitable concentration of PMPs may be used to ensure sufficient binding of the PMPs to the target (e.g. formation of a sufficient number of target-PMP complexes. For lyophilized PMP formulations, any suitable weight of lyophilized product may be used to ensure the proper concentration of PMPs to be mixed with the biological sample. For liquid formulations, the liquid composition comprising the PMPs may comprise any suitable concentration of PMPs to ensure sufficient binding of the PMPs to the target (e.g. formation of a sufficient number of target-PMP complexes). For example, PMPs may be present in the liquid composition at about 1-20% (v/v). For example, PMPs may be present in the liquid composition in an amount of about 1%, 2%, 3%, 4%, 5%, 6%, 7%, 8%, 9%, 10%, 11%, 12%, 13%, 14%, 15%, 16%, 17%, 18%, 19%, or about 20% (v/v).

In some embodiments, the liquid composition comprising PMPs contains other suitable reagents for processing/handling of biological samples. For example, the liquid composition comprising PMPs may contain one or more detergents, reducing agents, buffers, inhibitors, enzymes (e.g. proteases), denaturants, etc. Any additional reagents (such as those described above) present in the biological sample may additionally be present in the liquid composition comprising PMPs. For example, the liquid composition may further comprise one or more reagents to decrease viscosity of the biological sample. For example, the liquid composition may comprise PMPs and DTT. The liquid composition may comprise other suitable buffers, inhibitors, and the like to prevent degradation of the target (e.g. target nucleic acid, target protein, etc.) during sample processing. Suitable inhibitors that may be present in the liquid composition comprising PMPs include, for example, RNase inhibitors, protease inhibitors, nuclease inhibitors, and the like. Lyophilized PMP formulations may contain other suitable reagents commonly used in the lyophilization process, including bulking agents, stabilizers, and other suitable excipients.

In some embodiments, the PMPs are mixed with the biological sample after the sample is aspirated into the pipette tip. For example, the biological sample (including a pre-treated biological sample) may be aspirated into a pipette tip, and mixed with PMPs (e.g. a liquid composition comprising PMPs) by pipetting up and down multiple times. In other embodiments, the biological sample may be aspirated into a pipette tip containing lyophilized PMPs, and thoroughly mixed with the lyophilized PMPs by pipetting up and down multiple times. In still other embodiments, the PMPs may be added to the biological sample to generate a composition comprising a plurality of target-PMP complexes, and a suitable volume of the composition is subsequently aspirated into the pipette tip. For example, the biological sample could be pre-treated by a suitable heat inactivation step, cell lysis step, freezing, etc. within a container, and the PMPs (e.g. PMPs contained in a liquid or lyophilized form) could be added to said container to create a composition comprising a plurality of target-PMP complexes. After suitable mixing (e.g. by pipetting up and down multiple times), the composition (e.g. composition containing the plurality of target-PMP complexes) could be aspirated into a pipette tip. In some embodiments, a suitable volume of the liquid composition comprising PMPs is present in the sample collection container at the time the sample is collected. For example, a subject may provide a saliva sample into a collection container already holding the liquid composition comprising PMPs. In some embodiments, a suitable amount of lyophilized PMPs are already present in the sample collection container at the time the sample is collected.

In some embodiments, the methods further comprise generating a liquid/air interface proximal to the bottom opening of the pipette tip. The liquid/air interface is an interface between the composition contained therein (e.g. the biological sample mixed with the PMPs), and the air. In some embodiments, the liquid/air interface is generated by further aspirating the composition within the pipette tip (e.g. while the bottom opening of the pipette tip is exposed to air), thus generating a pocket of air at the bottom opening of the pipette tip. The pocket of air is also referred to herein as an air-gap. In other embodiments, such as embodiments wherein the liquid is aspirated through a side opening of the pipette tip, the liquid/air interface is generated inherently during aspiration of the liquid through the side opening. In some embodiments, the pocket of air generated is 2-30 mm high. For example, the pocket of air may be 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, or 30 mm high. The appropriate height and/or volume of the pocket of air may be selected based on the diameter of the opening of the pipette tip, the temperature of the biological sample, the viscosity of the biological sample, the nature of the paramagnetic particles used, the strength of the magnetic force, etc.

In some embodiments, the methods further comprise applying a first magnetic force to the composition (e.g. the composition comprising the target-PMP complexes) to generate a concentration of target-PMP complexes proximal to the liquid/air interface. For example, a multichannel pipette containing multiple pipette tips holding the composition may be moved in proximity to a first magnet. Alternatively, a first magnet may be moved in proximity to the multichannel pipette containing the multiple pipette tips. The first magnet attracts the PMPs within the composition, thus pulling target-PMP complexes towards the first magnet. The first magnet is therefore also referred to herein as a “preconcentration magnet”. The location and strength of the preconcentration magnet in relation to the pipette tip determines the area within the pipette tip at which the target-PMP complexes will collect. Accordingly, the preconcentration magnet should be appropriately placed relative to the pipette tip to ensure that the target-PMP complexes collect proximal to the liquid/air interface. In some cases, the preconcentration magnet is brought into proximity of the pipette (e.g., on a manual pipette fitted with a moveable or removable magnet) while in other cases, the pipette is brought into proximity of the magnet (e.g., as might occur using a liquid handling robot).

Following generating the concentration of target-PMP complexes proximal to the liquid/air interface at the bottom opening of the pipette tip, in some embodiments, the methods further comprise drawing the target-PMP complexes through the liquid/air interface and into a suitable sample collection device. Accordingly, the methods may further comprise applying a second magnetic force to the composition containing the target-PMP complexes. The second magnetic force may be a second magnet (i.e. a separate magnet from the preconcentration magnet). In some embodiments, the second magnetic force may be stronger than the first magnetic force. For example, the first magnetic force may be of sufficient strength to draw the target-PMP complexes towards the magnet but not strong enough to pull the target-PMP complexes through the liquid/air interface. In contrast, the second magnetic force is of sufficient strength to achieve purification of the target-PMP complex by drawing the target-PMP complex through the liquid/air interface. Drawing the target-PMP complex through the liquid/air interface represents a facile means for purifying the target from the biological sample without the need for additional processing steps (e.g. wash steps) that may result in sample loss or contamination with unwanted components from the biological sample itself.

The second magnet may be positioned in a suitable location such that the target-PMP complexes are drawn through the liquid/air interface and into a sample collection device. The second magnet is therefore also referred to herein as a “sample collection magnet”. For example, the sample collection magnet may be positioned below the sample collection device. In some embodiments, the sample collection device is a multi-well plate. In such embodiments, the second magnet may be positioned below the multi-well plate.

Any desired sample collection device may be used. Examples include, but are not limited to, test tubes, microcentrifuge tubes, dishes, slides, plates, multi-well plates (e.g., 4-well, 8-well, 12-well, 96-well, 384-well, etc.), flasks, vials, channels, and the like. Other suitable examples include porous materials, such as filter paper. Drawing the target-PMP complexes “into” a sample collection device is meant to encompass any means by which the target-PMP complexes are brought into contact with the sample collection device or a surface thereof. For example, drawing the target-PMP complexes onto a piece of filter paper, onto a plate, etc. is meant to be encompassed by drawing the target-PMP complexes “into” a sample collection device.

For automated, high-throughput systems and methods multi-well plates used in combination with multi-pipette arrays are particularly useful.

In some embodiments, the sample collection device comprises a wash buffer. For example, in particular embodiments the distinct wells on the multi-well plate comprise a wash buffer. In some embodiments, the wash buffer comprises water. In some embodiments, the wash buffer comprises ethanol. In some embodiments, a layer of oil (e.g. light mineral oil) may reside above the wash buffer. Accordingly, the target-PMP complexes will be drawn through the layer of oil prior to contacting the wash buffer, thus further purifying unwanted contaminants from the target-PMP complexes. In some embodiments, the methods described herein further comprises aspirating the wash buffer (and the layer of oil, if present) from the sample collection device (e.g. wells) while the device is positioned above the magnet such that the target-PMP complexes remain, and allowing the target-PMP complexes to dry. In some embodiments, additional wash steps may be performed. In such embodiments, following removal of the wash buffer reagents for the detection of the target may be added to the sample collection device, and suitable methods for detection of the desired pathogen may be performed. In some embodiments, the sample collection device comprises reagents for detection of the target.

In some embodiments, reagents for detection of the target comprise reagents for nucleic acid amplification (e.g. PCR, isothermal amplification, and the like) and/or sequencing. In some embodiments, the reagents for detection of the target comprise reagents for loop-mediated isothermal amplification (LAMP)-based detection of the target. In general, LAMP reactions include a DNA polymerase with strong strand displacement activity and tolerance for elevated temperatures and up to six DNA oligonucleotides of a certain architecture. RT-LAMP reactions additionally include a reverse transcriptase. Samples with potential template molecules are added to the reaction and incubated for 20 to 60 min at a constant temperature (e.g. 65° C.). The oligonucleotides act as primers for the reverse transcriptase, and additional oligonucleotides for the DNA polymerase are designed so the DNA products loop back at their ends. These, in turn, serve as self-priming templates for the DNA polymerase. In the presence of a few RNA template molecules, a chain reaction is set in motion, which then runs until the added reagents (in particular, the deoxynucleotide triphosphates) are used up.

LAMP assays or RT-LAMP assays may be a preferred embodiment due to their rapid nature, one-tube processing, and easy visualization and/or display of results without the need for expensive equipment or additional materials. In particular embodiments, the reagents for detection of the target comprise reagents for a colorimetric assay for detecting the target. Such embodiments allow for a facile visualization of whether or not the sample contains the target of interest. In some embodiments, the sample collection device contains reagents for a colorimetric loop mediated isothermal amplification (LAMP) assay. In embodiments wherein the nucleic acid is RNA, the sample collection device may contain reagents for a colorimetric RT-LAMP assay. In some embodiments, the reagents for a colorimetric LAMP assay (or colorimetric RT-LAMP assay) further include an indicator, which permits evaluation of a color change in the sample in the presence of sufficient nucleic acid (e.g. the target nucleic acid which the LAMP or RT-LAMP reagents are designed to detect). Suitable indicators include pH-sensitive indicators and metal-sensitive indicators. In preferred embodiments, pH-sensitive indicators (e.g. phenol red) may be used, due to their easy visualization with the naked eye.

In some embodiments, the reagents for detection of the target comprise reagents for a fluorescent assay for detecting the target. For example, the sample collection device may contain reagents for a fluorescent LAMP or fluorescent RT-LAMP assay. Any suitable fluorescent dye may be used in a fluorescent LAMP or fluorescent RT-LAMP assay to permit a fluorescent signal to be generated in the presence of sufficient nucleic acid.

In some embodiments, the reagents comprise oligonucleotides (e.g. primers) designed for detection of bacterial nucleic acid. In some embodiments, the reagents comprise oligonucleotides designed for detection of viral RNA. For example, the reagents may comprise oligonucleotides designed for detection of a viral upper respiratory infection selected from SARS-CoV2, SARS, a coronavirus, rhinovirus, influenza, respiratory syncytial virus, etc. In some embodiments, the reagents comprise oligonucleotides for detection of SARS-CoV-2 RNA. In some embodiments, the reagents comprise oligonucleotides designed for detection of viral DNA, or DNA from bacteria, as well as reagents for the detection of other microbes, including prions using a protein misfolding cyclic amplification assay, for example.

In some embodiments, protein detection assays are used, e.g., ELISAs, RIAs, etc., which may be quantitative, semi-quantitative, or qualitative. Serology assays to detect IgM, IgG or total antibodies may also be used to evaluate past exposure and immune status, for example.

In some embodiments, the reagents for detection of the target and/or the wash buffer are retained on a bottom surface of the sample collection device (e.g. multi-well plate) by a sealant. Suitable sealants include, for example, oils that are liquid at room temperature. For example, the sealant may comprise mineral oil. For example, a layer of mineral oil may float above the reagents for detection of the target. As another example, a layer of mineral oil may float above the wash buffer. The target may be drawn through the liquid/air interface and subsequently pass through the air/oil interface prior to contacting the wash buffer and/or detection reagents contained within the sample collection device.

In some embodiments, the biological sample is obtained from a subject suspected of having an infection. The subject may be suspected of having any infection by a pathogen that can be detected in the saliva of the subject, or an infection which causes the subject to produce antibodies which may be detected in saliva of the subject. In some embodiments, the subject is suspected of having SARS-CoV2, coronavirus, rhinovirus, influenza, respiratory syncytial virus, adenovirus, parainfluenza, human immunodeficiency virus, human papillomavirus, rotavirus, hepatitis C virus, zika virus, Ebola virus, tuberculosis, Borrelia burgdorferi, Staphylococcus, aspergillus, or Streptococcus pyogenes. In some embodiments, the subject may be suspected of having a bacterial infection or a viral infection. For example, the subject may be suspected of having an upper respiratory infection. For example, the subject may be suspected of having a viral upper respiratory infection, including infection with SARS-CoV-2, a coronavirus, rhinovirus, influenza, respiratory syncytial virus, and the like.

In some embodiments, the methods described herein are performed on a single biological sample. In other embodiments, the methods are performed simultaneously on a plurality of biological samples. For example, methods for simultaneously assessing a plurality of biological samples involve aspirating each biological sample is aspirated into a separate pipette tip, and performing the tasks described herein on each pipette tip simultaneously. Such embodiments may be performed using a multichannel pipette.

In some embodiments, biological samples may be pooled and subsequently used in the methods described herein. For example, biological samples may be collected from a plurality of distinct individuals, pooled together, and used in the methods described herein to determine whether a population has cases of infection with a pathogen (e.g. with SARS-CoV2). As another example, a plurality of biological samples may be collected from an individual, and the plurality of biological samples from a distinct individual may be pooled to increase the amount of sample available to be used in the methods described herein. Such embodiments may be useful for instances where an individual may be unable to provide an adequate volume of saliva during one collection, or when multiple tests may be performed using the same sample.

In some embodiments, a single pipette tip (e.g., a pipette tip containing a sample collected from one individual) may be used to aspirate and subject portions of the composition contained therein (e.g., the composition comprising the plurality of target-PMP complexes) to the magnet-based purification process described herein multiple times. For example, a portion of composition can be aspirated into the pipette tip (such as a biological sample obtained from a single individual) and placed above a magnet, wherein the magnet is placed below a sample collection device such that the target-PMP complexes contained therein are drawn into the sample collection device. The pipette tip and/or sample collection device may be moved, and a second portion of the target-PMP complexes can be aspirated into the pipette tip from the diluted biological sample and then drawn into a second sample collection device, For example, the sample collection device may be a multi-well plate. The first portion of the target-PMP complexes may be drawn into a first well, the second portion may be drawn into a second well, a third portion may be drawn into a third well, etc., until the desired number of distinct wells containing target-PMP complexes are obtained. In this manner, the assay may be multiplexed such that a single diluted biological sample provides sufficient target-PMP complexes for performing multiple target detection assays.

In some embodiments, the methods described herein are automated. For example, the method may be executed by a computer, wherein the computer comprises a processor and a memory. The memory may contain software which instructs the processor to execute a given task. For example, the memory may contain software which instructs the processor to cause a multichannel pipette to attach pipette tips to the pipette, aspirate a biological sample, mix the biological sample with PMPs to generate a composition comprising a plurality of target-PMP complexes within a pipette tip, position the pipette tip appropriately relative to a preconcentration magnet, position the pipette tip appropriately relative to a sample collection magnet, and other necessary functions to perform the claimed method. As another example, the memory may contain software which instructs the processor to cause a moveable surface housing one or more components (e.g. sample collection devices, mixing containers, etc.) to move to a desired position. Instruments may be used that contain one or more robotic components that permit movement of system components to facilitate automation of some or all of the steps of the method.

In some aspects, provided herein are systems for automated isolation of a target from a plurality of biological samples. The systems comprise a plurality of pipette tips, a multichannel pipette, and an apparatus for operating the multichannel pipette. In some embodiments, the apparatus for operating the multichannel pipette is configured to permit horizontal and/or vertical movement of the multichannel pipette. For example, the apparatus may comprise a horizontal rod that permits the multichannel pipette to slide in a planar fashion along the length of the rod. In such embodiments, the apparatus for operating the multichannel pipette facilitates movement of the multichannel pipette horizontally along the length of the rod. In some embodiments, the apparatus for operating the multichannel pipette comprises a vertical rod, or an expandable member attached to the multichannel pipette that expands and contracts, thus permitting vertical movement of the multichannel pipette. For example, the apparatus may comprise a vertical rod that permits the multi-channel pipette to slide in a vertical fashion along the length of the rod. In such embodiments, the apparatus for operating the multichannel pipette facilitates movement of the multichannel pipette up or down. Such up and down movement of the multichannel pipette may facilitate attachment of pipette tips thereto, and may facilitate proper placement of the pipette tips contained on the multichannel pipette within a container (e.g. placement within a multi-well plate, placement within a sample collection device, and the like).

In some embodiments, the system further comprises a moveable surface. One or multiple components of the system may be placed on the moveable surface. The moveable surface may change orientation and may move in the x-y plane and/or in the vertical z-direction. Such movement may facilitate proper positioning of one or more components on the surface relative to the multichannel pipette.

In some embodiments, a plurality of pipette tips contain lyophilized paramagnetic particles. In some embodiments, the plurality of pipette tips are held within a suitable pipette tip container. The pipette tip container may be placed on a moveable surface. For example, the pipette tips may be held in a container placed on a moveable surface, and the moveable surface may be positioned to align the plurality of pipette tips with the multichannel pipette. Following proper alignment, the moveable surface and/or the multichannel pipette may be moved vertically (e.g. up or down) to permit attachment of the pipette tips to the multichannel pipette.

In some embodiments, the systems comprise a plurality of biological samples. For example, the systems may comprise a plurality of biological samples, wherein each biological sample is held within a suitable sample container. For example, the sample container may be a multi-well plate. The biological samples may be pre-treated by heating and/or treatment with a viscosity reducing agent, as described above. In some embodiments, the plurality of biological samples are held within a container (e.g. a first multi-well plate) which is placed upon a moveable surface. The moveable surface and/or the multichannel pipette (e.g. containing a plurality of pipette tips) may move in a suitable fashion to facilitate alignment of the pipette tips with the plurality of biological samples. The apparatus for operating the multichannel pipette may aspirate the biological samples, such that each biological sample is aspirated into a different pipette tip. In other embodiments, pooled samples are tested.

In some embodiments, the systems described herein further comprise a mixing container for combining and subsequently mixing biological samples with PMPs. For example, the mixing container may be used for combining and subsequently mixing biological samples with lyophilized PMPs or with a liquid composition comprising paramagnetic particles. In some embodiments, the mixing container comprises a multi-well plate. In some embodiments, a plurality of wells in the multi-well plate contain a liquid composition comprising PMPs, such that a plurality of biological samples may be mixed with the plurality of liquid compositions comprising PMPs simultaneously. In some embodiments, the apparatus for operating the multichannel pipette injects the liquid composition comprising PMPs into each of the plurality of wells prior to mixing the liquid composition with the biological samples. In other embodiments, a plurality of wells in the multi-well plate contain a lyophilized PMPs.

In some embodiments, the mixing container is located on a moveable surface. In some embodiments, the apparatus for operating the multichannel pipette and/or the moveable surface are configured to position the multichannel pipette such that the plurality of pipette tips are aligned with a plurality of wells in the mixing container (e.g. multi-well plate), such that the biological samples may be mixed with the PMPs contained therein. The apparatus for operating the multichannel pipette may facilitate aspiration and injection of liquid by the multichannel pipette, such that the biological samples and the PMPs may be mixed. Mixing of the biological samples with the PMPs results in formation of target-PMP complexes.

In some embodiments, the apparatus for operating the multichannel pipette is configured to create a liquid/air interface at the bottom opening of the plurality of pipettes attached thereto. For example, the apparatus may be configured to further aspirate the composition contained therein following formation of target-PMP complexes (e.g. after mixing with the PMPs) within the pipette tips. Such further aspiration creates an air pocket at the bottom opening of the pipette tips, such that a liquid/air interface exists between the meniscus of the liquid contained within the pipette tip and the pocket of air at the bottom opening.

In some embodiments, the system further comprises a first magnet. The first magnet may be of suitable strength to pull the target-PMP complexes to a desired area within the pipette tip. For example, the first magnet may be of suitable strength to pull the target-PMP complexes towards the bottom of the pipette tip to form a concentration of target-PMP complexes proximal to the bottom opening of the pipette tip. The first magnet may therefore also be referred to herein as a “preconcentration magnet”. In some embodiments, the preconcentration magnet may be placed on a moveable surface. The apparatus for operating the multichannel pipette and/or the moveable surface may position the multichannel pipette appropriately with relation to the preconcentration magnet to achieve formation of the concentration of target-PMP complexes in the desired location.

In some embodiments, the system further comprises a sample collection device. For example, the sample collection device may be a multi-well plate. The sample collection device (e.g. multi-well plate) may contain reagents for detection of the target in the plurality of samples. For example, a plurality of wells in a multi-well plate may contain reagents for detection of the target in the plurality of samples. In some embodiments, the apparatus for operating the multichannel pipette is configured to inject the reagents for detection of the target into a plurality of wells in the multi-well plate. In some embodiments, the sample collection device is housed on a moveable surface. The moveable surface and/or the multichannel pipette may move to align each pipette tip on the multichannel pipette with a separate well in the sample collection device (e.g. multi-well plate).

In some embodiments, the system further comprises a second magnet. The second magnet is used to draw the target-PMP complexes through the liquid/air interface and into the sample collection device, and may therefore also be referred to herein as the “sample collection magnet”. In some embodiments, the sample collection magnet is stronger than the preconcentration magnet. The sample collection magnet may be of suitable strength to draw the target-PMP complexes through the liquid/air interface at the bottom opening of each pipette tip and into the sample collection device. However, the sample collection magnet does not need to be stronger than the preconcentration magnet. In some embodiments, proximity may be employed rather than increased strength to effectuate the movement of target-PMP complexes through the air gap. For example, the preconcentration magnet and the sample collection magnet may be of the same strength, but the sample collection magnet may be placed in closer proximity to the pipette tip in order to draw the target-PMP complexes contained therein through the liquid/air interface. In some embodiments, the sample collection device is a multi-well plate. The sample collection magnet may be positioned at a suitable location to draw the target-PMP complexes into the sample collection device. For example, the sample collection magnet may be positioned below the multi-well plate. In some embodiments, the apparatus for operating the multichannel pipette and/or a moveable surface housing the multi-well plate is configured to position the multichannel pipette above the multi-well plate, such that each pipette tip is aligned with a well in the multi-well plate, thus permitting target-PMP complexes present in each pipette tip to be pulled through the liquid/air interface by the second magnet and into a well. In some embodiments, the system comprises a plurality of sample collection magnets (e.g. arranged in an array). For example, a plurality of sample collection magnets may be used to address a plurality of pipette tips and/or pipettes at the same time or in sequence. In some embodiments, a third set of magnets are used to influence or adjust the uniformity and strength of the sample collection magnets. For example, when the sample collection magnets are arranged in an array pattern, a third set of magnets positioned around the perimeter of the array to reduce edge effects, maintaining a more consistent magnetic field for each sample collection magnet in the array. Accordingly, the third set of magnets may be referred to herein as “field stabilization magnets”.

In some embodiments, the apparatus for operating the multichannel pipette is configured to aspirate and/or inject liquid. In particular embodiments, the apparatus for operating the multichannel pipette is configured to:
a. Mix the biological sample with paramagnetic particles (PMPs) to generate a composition comprising a plurality of target-PMP complexes within a pipette tip, and
b. Generate a liquid/air interface proximal to a bottom opening of the pipette tip.

In particular embodiments, the apparatus for operating the multichannel pipette is configured to:
a. Mix the biological sample with paramagnetic particles (PMPs) to generate a composition comprising a plurality of target-PMP complexes,
b. Aspirate the composition into a pipette tip through a bottom opening of the pipette tip; and
c. Generate a liquid/air interface proximal to the bottom opening of the pipette tip.

In particular embodiments, the apparatus for operating the multichannel pipette is configured to:
a. Mix the biological sample with paramagnetic particles (PMPs) to generate a composition comprising a plurality of target-PMP complexes,
b. Position a pipette tip such that a bottom opening of the pipette tip is in conformal contact with a surface, such that liquid is unable to enter the pipette tip through the bottom opening, and
c. Aspirate the composition into a pipette tip through a side opening of the pipette tip, thereby generating a liquid/air interface proximal to the bottom opening of the pipette tip.

In particular embodiments, the apparatus for operating the multichannel pipette is configured to perform the following steps, in order:
a. Aspirate biological samples into pipette tips attached to the multichannel pipette;
b. Pipette biological samples into mixing container holding paramagnetic particles;
c. Mix the biological samples with the PMPs to generate a composition comprising a plurality of target-PMP complexes within the pipette tips; and
d. Further aspirate the composition within the pipette tips to generate a liquid/air interface at a bottom opening of each pipette tip.

In some embodiments, the apparatus for operating the multichannel pipette is configured to move the multichannel pipette (e.g. change orientation and/or move the multichannel pipette in the x-y plane and/or in the vertical z-direction). In particular embodiments, the apparatus for operating the multichannel pipette is configured to perform one or more of the following: a. Position the multichannel pipette above a container holding a plurality of pipette tips;
b. Move the multichannel pipette downward to attach a plurality of pipette tips to the multichannel pipette;
c. Position the multichannel pipette above a mixing container;
d. Move the multichannel pipette downward to permit contact between the plurality of pipette tips and the contents within the mixing container;
e. Position the multichannel pipette proximal to a preconcentration magnet;
f. Position the multichannel pipette proximal to a sample collection magnet.

In some embodiments, the apparatus for operating the multichannel pipette is further configured to aspirate and/or inject liquid, including wash buffer, reagents for detection of a target, etc.

In some embodiments, the apparatus for operating the multichannel pipette is further configured to move the multichannel pipette to a position above a waste basin and eject used pipette tips into the waste basin.

In some embodiments, the system further comprises a computer. For example, the apparatus for operating the multichannel pipette may be controlled by a computer. For example, the computer may dictate movement of the multichannel pipette (e.g. movement along a horizontal or vertical rod) and instruct the apparatus for operating the multichannel pipette to induce aspiration/injection of liquid by the multichannel pipette. As another example, the moveable surface may be controlled by a computer. In some embodiments, the apparatus for operating the multichannel pipette and the moveable surface are both controlled by a computer.

The computer may comprise a processor and a memory. The memory may contain software which instructs the processor to execute a given task. For example, the memory may contain software which instructs the processor to cause the apparatus for controlling the multichannel pipette to perform a variety of tasks related to the methods described herein. For example, the memory may contain software which instructs the processor to cause the apparatus for controlling the multichannel pipette to perform one or more of the following tasks: attach pipette tips to the pipette, aspirate a biological sample into the pipette, position the biological sample above a first multi-well plate, mix the biological sample with PMPs contained within the first multi-well plate to generate a composition comprising a plurality of target-PMP complexes, position the pipette tips appropriately relative to a preconcentration magnet, position the pipette tips appropriately relative to a sample collection magnet, position the multi-channel pipette above a waste bin, aspirate and/or inject liquids including wash buffer and reagents for detection of a target, and eject used pipette tips.

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

### EXAMPLES

### Example 1

### Reagents, Standards, Supplies, and Equipment

### Reagents and Standards:

NEB WarmStart LAMP Kit (DNA & RNA)—NEB catalog E1700. Store at −15 to −25° C. through the kit expiration date. Avoid repeated freezing and thawing. Kits include the following reagents:
i. WarmStart LAMP 2× Master Mix
ii. Fluorescent dye (50×)

10× stock of primers specific to SARS-CoV-2 Genes N and As1e—Lyophilized primers were purchased from IDT and reconstituted with nuclease free ddH₂O to a final concentration of 100 μM. Stored at −15 to −25° C. through the expiration date. Primers were then mixed to a final concentration:
i. F3—2 μM
ii. B3—2 μM
iii. FIP—16 μM
iv. BIP—16 μM
v. LF—4 μM
vi. LB—4 μM

SeraSil-Mag Silica Coated Superparamagnetic Bead Mixture—Mixed thoroughly before use. Stored at ambient temperature.

Bleach, minimum 5% or 0.7M sodium hypochlorite solution

Phosphate-Buffered Saline (PBS) pH 7.4—Gibco catalog #10010-023 or equivalent. Stored at ambient temperature.

Sample Buffer—10 mM DTT and 0.5% Triton x-100 in PBS (pH 7.4) Stored at ambient temperature.

Wash Buffer—Stored at ambient temperature.

RNase-Free, DNase-Free Water—Invitrogen catalog AM9937(or equivalent). Stored at ambient temperature.

Light Mineral Oil—Millipore Sigma catalog #330779 or equivalent. Stored at ambient temperature.

SARS-CoV-2 Positive controls—Prepared aliquots of a diluted inactivated (heat or irradiation) SARS-Cov-2 virion in PBS (pH 7.4). High, medium, low, and negative controls consist of viral loads at 1.0×10⁵, 1.0×10⁴, 1.0×10³, and 0 copies/mL. Stored at <−70° C.

### Supplies

Personal Protective Equipment (PPE): Latex free Nitrile gloves, pre-amplification lab coat and safety glasses

10% (v/v) bleach (0.5% (w/v) sodium hypochlorite) made fresh daily and 70% ethanol

Biohazard disposal containers (for sharps and non-sharps)

1.5 mL polypropylene screw cap tubes, sterile, non-siliconized, and conical (Sarstedt 72.693.105 or equivalent)

Sterile disposable serological pipets, 10 and 25 mL

Sterile fine-tip transfer pipettes, RNase-free

Wypalls

Pipette tips (capacity 1000 μL, 200 μL, 20 μL, 10 μL) plugged (aerosol barrier). Plugged (aerosol barrier) tips must be used to prevent reagent and amplicon cross contamination

RNAse Away, Fisher 14-375-35, 11, Store at ambient temperature.

### Equipment

Pre-Amplification Area
Gilson Pipetmax liquid handling robot
On stage magnetic arrays for Pipetmax
On stage tip box holders for Pipetmax
Networked Personal Computer with Trilution 3.0, R studio programs, and other software that, among other functions, is designed for use in controlling the communication between components, routing data to the cloud, etc., and peripherals
Pipettes (capacity 1000 μL, 200 μL, 20 μL, 10 μL, multi and single channel). Pipettes should be accurate within 3% of stated volume.
−20° C. Reagent Freezer
−80° C. Specimen Freezer
2-8° C. Refrigerator
Oven capable of reaching 65° C.
Vortex mixer
Centrifuge (e.g. Eppendorf 5804 R)
BL2+ Biological safety cabinet with integrated germicidal ultraviolet light
Milli-Q Water Purification System

### Procedure

The following describes the steps of mixing a biological sample with paramagnetic particles to generate a composition comprising target-PMP complexes, generating a liquid/air interface proximal to the bottom opening of a pipette tip and applying a magnetic force to the composition to generate a concentration of target-PMP complexes proximal to the liquid/air interface. Application of a magnetic force to the composition to draw target-PMP complexes through the liquid/air interface and into a sample collection device is accomplished with a magnetic array oriented beneath the reaction plate. The Gilson Pipet Max automated pipetting system was used, but other robotic pipette or high precision liquid handling systems may be used.

Summary of Automated steps
1.) Once all reagents, tip boxes, and plates were placed and secured on the deck of the PipetMax, the user closes the safety protective shield over the PipetMax and then pressed “run”
2.) The PipetMax performed a series of pre-dispense steps whereby, wash buffer and oil were added to the reaction plate and sample buffer and PMPs were added to the Mixing Plate
3.) Next the PipetMax ensured that all sample collection tubes or wells in the prepared sample source plate contained equal volumes of sample by “levelling”, or by pipetting sample away.
4.) The following describes the step of mixing a biological sample with paramagnetic particles to generate a composition comprising target-PMP complexes. The Pipetmax added sample buffer to each sample, mixed by pipetting up and down, and then transferred the diluted sample to the mixing plate where the sample was then mixed with PMPs to initiate creation of target-PMP complexes.
5.) After enough time was allowed for target-PMP binding the PipetMax then aspirated the composition into a pipette tip through a bottom opening of a pipette tip. The pipette head raised up, so the bottom orifice is no longer submerged in liquid, and a small amount of air was aspirated into the tip, generating a liquid/air interface proximal to the bottom opening of the pipette tip.
6.) Once the composition of target-PMPs wqs contained within the pipette tip, a magnetic force was applied to the composition to generate a concentration of target-PMP complexes proximal to the liquid/air interface.
7.) Next, the PipetMax plunged the pipette tip containing the pre-concentrated target-PMPs into the reaction plate, thereby pinning the air bubble at the opening of the pipette tip between the target-PMP composition and the wash buffer. Application of a magnetic force to the composition to draw target-PMP complexes through the liquid/air interface and into a sample collection device occurred here due to the magnetic array oriented beneath the reaction plate.
8.) Lastly, the Pipetmax aspirated and disposed of the wash buffer and added LAMP reaction reagents to the target-PMPs.

### SARS-CoV-2 Isothermal LAMP by Air Purification Assay(Pre-PCR Room) Preparation of Sample Buffer (Biosafety Cabinet)

33.851 mL of PBS was pipetted into a 50 mL conical tube. 229.7 μL 1% Triton x-100 was added. 459.4 μL 1M DTT was added. The tube was inverted 10-20×.

### Preparation of LAMP MasterMix (Static Airhoods Clean Room)

The sterile hood, pipettes, and surrounding work area was wiped down with 10% bleach. Master mixes were set up on a cold block. Each reagent was gently mixed and microfuged prior to pipetting. Two clean 5.0 mL eppendorf tubes were labeled as “Covid” and “RNase P”. Using a clean tip for each reagent, the volume calculated from Table 2 (below) was added for each of the components comprising the master mix for each primer set. The freshly prepared master mix was vortexed and microfuged. Mixes were kept on ice until the Gilson robot was ready to add LAMP mixture.

TABLE 1
Overview of Sample Preparation

| | # of Samples | | |
|---|---|---|---|
| (μL) | 16 | 32 | 48 |
| 2x Master Mix | 425 | 625 | 825 |
| 10x Primer Stock | 85 | 125 | 165 |
| Fluorescent Dye | 17 | 25 | 33 |
| Nuclease-Free Water | 323 | 475 | 627 |
| Total Volume | 850 | 1250 | 1650 |

TABLE 2
LAMP Master Mix Recipes

| | Sample | NF | | | Covid | Rnase P |
|---|---|---|---|---|---|---|
| # of | Buffer | Water | Mineral | Beads | LAMP | LAMP |
| Samples | (μL) | (μL) | Oil (μL) | (μL) | (μL) | (μL) |
| 16 | 1000 | 1000 | 1000 | 150 | 100 | 100 |
| 32 | 1000 | 1000 | 1000 | 150 | 150 | 150 |
| 48 | 1000 | 1000 | 1000 | 150 | 200 | 200 |

TABLE 3
Reagent Volumes to Add to Source Plate Per Well

| | 1 | 2 | 3 | 4 | 5 | 6 | 7 | 8 | 9 | 10 | 11 | 12 |
|---|---|---|---|---|---|---|---|---|---|---|---|---|
| A | Sample | Sample | Sample | Sample | Bead | NF- | Mineral | NF- | Mineral | Empty | COVID | Rnase P |
| B | Buffer | Buffer | Buffer | Buffer | Mixture | Water | Oil | Water | Oil | | LAMP | LAMP |
| C | (1 mL/ | (1 mL/ | (1 mL/ | (1 mL/ | (150 uL/ | (1 mL/ | (1 mL/ | (1 mL/ | (1 mL/ | | Mix | Mix |
| D | well) | well) | well) | well) | well) | well) | well) | well) | well) | | (190 uL/ | (190 u/ |
| E | | | | | | | | | | | well) | wellL) |
| F | | | | | | | | | | | | |
| G | | | | | | | | | | | | |
| H | | | | | | | | | | | | |

### Prepare Reagent Plate (Static Air Hoods/Laminar Flow Hood)

Each of the following sequential steps detailing the addition of reagents was conducted in one of the static air hoods/laminar flow hoods. Prior to entry into the hood, the depository was emptied and the ultraviolet light in the static air hood was turned on for 5 minutes. Following UV Sterilization, all of the surfaces and pipettes were wiped down with 10% Bleach.

The Reagent Source Plate was filled with the volumes found in Table 3. Briefly, 1 mL Sample Buffer was added to every well in columns 1-4 of the Reagent Plate. The bead solution was vortexed for 30 seconds, inverting intermittently, and 150 μL bead solution was added to every well in column 5 of the Reagent Plate. A 6× concentrated bead stock was created by transferring 7.2 mL of well-mixed bead mixture into a 15 mL conical tube. The beads were sedimented by placing a strong magnet (neodymium) on the side of the tube. Once the beads were collected near the magnet, the aqueous portion was slowly removed, being careful not to disturb the beads. The magnet was removed, and beads were resuspended in 1.2 mL of NF water. 1 mL nuclease-free water was added to every well in columns 6 and 8 of the Reagent Plate. 1 mL light mineral oil was added to every well in columns 7 and 9 of the Reagent Plate. A suitable volume from the tube labelled “Covid” was added to each of the wells in column 11 of the Reaction Plate. A volume from the tube labelled “RNase P” was added to each of the wells in column 12 of the Reaction Plate.

Reagents were returned to the −20° C. freezer, refrigerator, or room temperature location as appropriate. The now-filled Reagent Source Plate was placed into deck position #3 on the Gilson PipetMax.

Prepare Sample Source Plate (Laminar Flow Biosafety Cabinet)

Each of the following sequential steps detailing the preparation, heat inactivation, and addition of patient samples to the 96 well plate were conducted in one of the laminar flow biosafety cabinets. Prior to entry into hood, the tip depository was emptied and the ultraviolet light in the biosafety cabinet was turned on for 5 minutes.

For each of 48 patient samples, 150 μL of saliva sample was pipetted into different wells in every other column starting with column #1 of the Sample Source Plate (0.5 mL V-Bottom PP Plate, taller plate).

Prepare the Gilson PipetMax

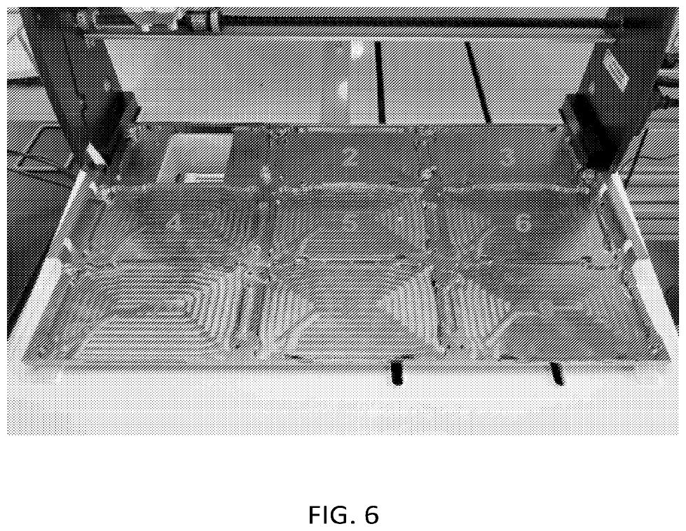

The layout of the Gilson PipetMax is shown in FIG. 6, which may be referred to herein for the specific positions described below.

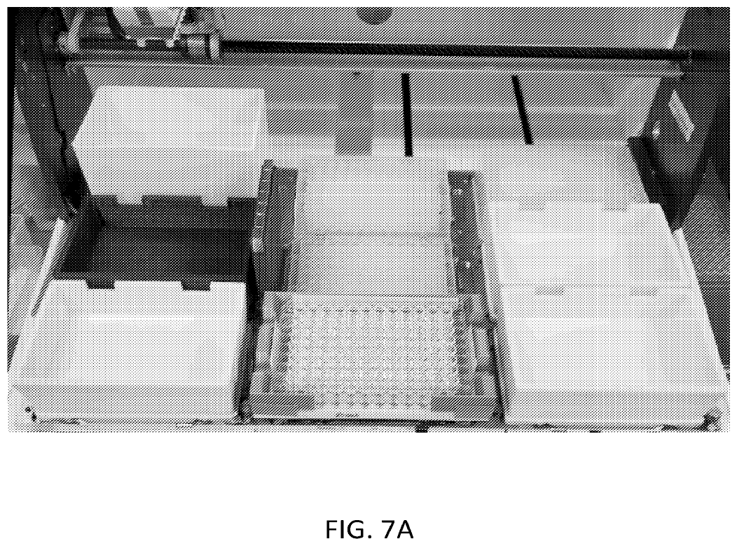

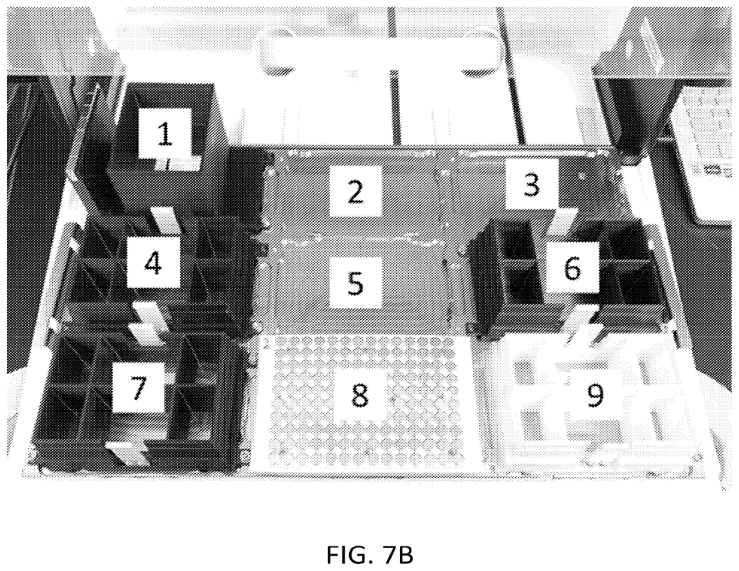

The Tip Disposal Box was placed onto deck position #1. The Reagent Source Plate was secured onto deck position #3. A new Mixing Plate was secured onto deck position #2. The filled Sample Source Plate was secured onto deck position #5, making sure that well A1 was oriented so as to be in the topmost left position of the deck position. A new Wash/Reaction Plate, without the lid, was placed on top of the magnet array (deck position #8) making sure that well A1 was oriented so as to be in the topmost left position of the deck position. The plastic wrap and the container lids were removed from 4 sets of tip boxes. Tip boxes were firmly pressed into the tip box cradles at positions #4, #6, #7, and #9. The final layout of the prepared Gilson PipetMax is shown in FIG. 7.

The air purification protocol described herein was performed in an automated fashion using a programmed computer. Prior to performing the protocol, it was ensured that all of the on-deck components were placed and oriented correctly, and that all of the non-consumable components (magnetic array, pre-concentration magnet, tip box cradles) were secured to the deck with the included fasteners.

Exemplary Air Purification/LAMP Protocol:

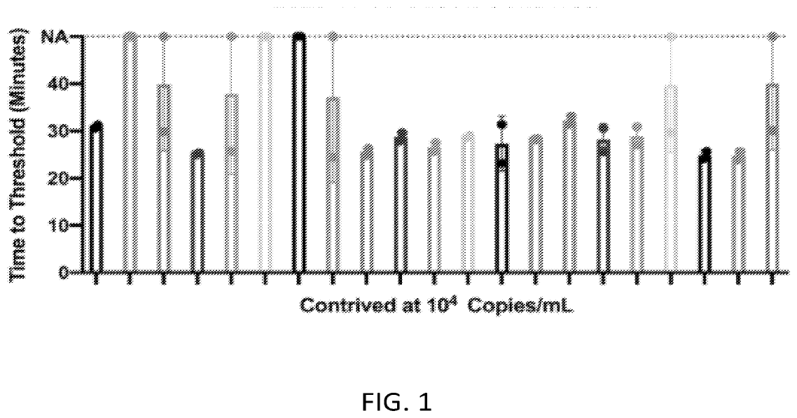

The appropriate volume of the LAMP mixture was added to each well of a multi-well plate (oil/water phases should separate appropriately automatically). The RNA:PMP complexes were purified from the sample by using a first magnet to generate a concentration of RNA:PMP complexes proximal to the opening of the pipette tips. A second magnet placed under the multi-well plate containing the wash buffer was used to draw the RNA:PMP complexes into the wells. The wash buffer was aspirated from each well and the target:PMP complexes were allowed to dry. The LAMP mixture was subsequently added to each well. LAMP was performed and results were analyzed using software to run desired algorithms to determine endpoints (e.g. positive, negative). Fluorescence was quantified using arbitrary fluorescent “read” intervals that generate a standard sigmoidal amplification curve. From this curve, proprietary R analysis calculates and can display time-to-threshold values. While the time-to-threshold values are useful for troubleshooting purposes, the results of LAMP are assessed as binary, wherein detection of a signal above threshold indicates a positive result (e.g. presence of the pathogen) and no detection or detection below threshold indicates a negative result. Time to threshold values for contrived samples are shown in FIG. 1. Results can also be displayed to the user by computer.

### Example 2

### Timing of DTT Addition

Efficacy sample extraction in terms of time to threshold for the LAMP assay was evaluated in samples with DTT added before heating the saliva and DTT added after heating the saliva, respectively. Samples were treated as generally described above. Saliva samples were diluted in Triton X-100 (0.005%), DTT (10 mM), paramagnetic beads (Cytiva beads, 10 μL/well), and PBS for a total volume of 420 μL. The wash volume was 160 μL. For the LAMP reaction, 12.5 μL master mix, 2.5 μL primers (to the N-gene and Asle of SARs-CoV2), and 0.5 μL dye were used along with 7 μL H₂O for a total volume in each well of 25 μL.

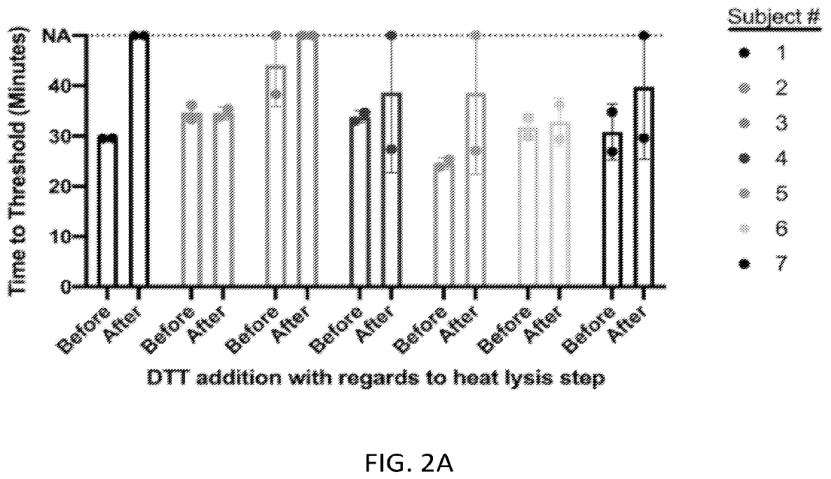

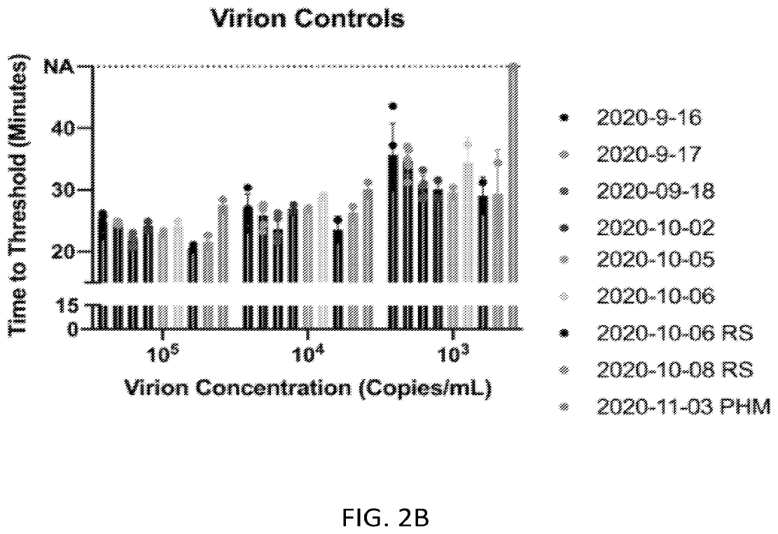

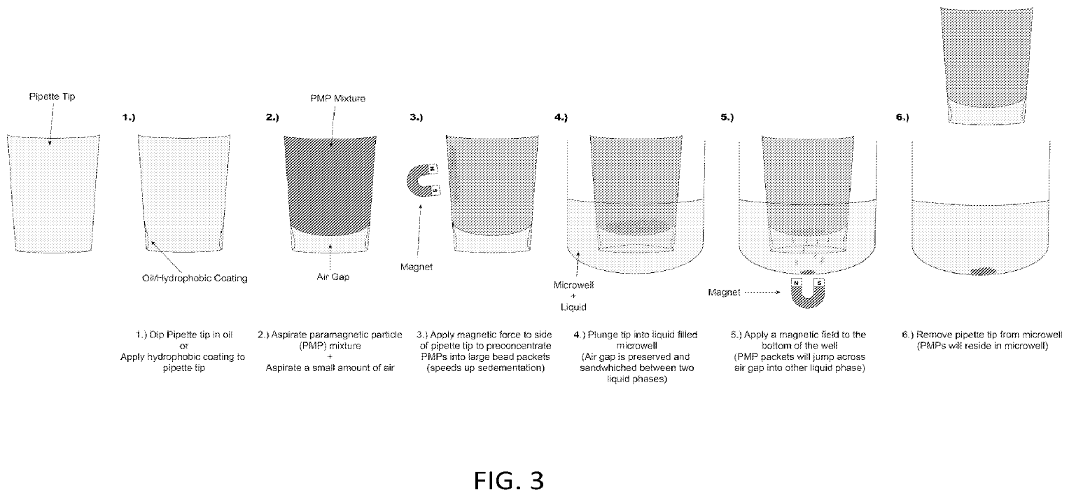

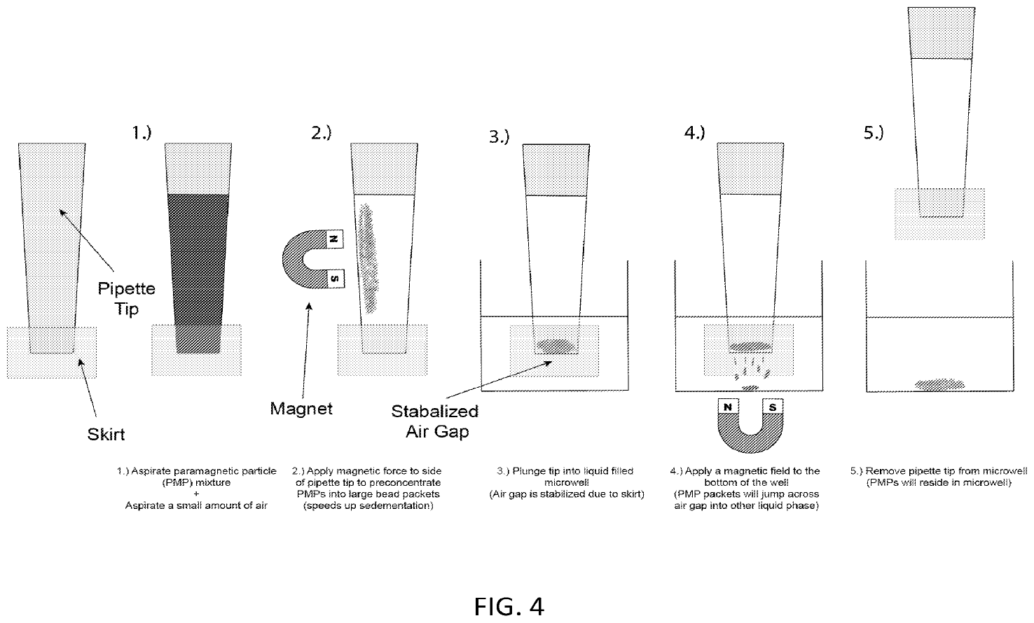

Results comparing timing of DTT addition are shown in FIG. 2A. Results show that addition of DTT before the heat lysis step decreases the time to threshold values for each sample. FIG. 2B shows comparative results from virion controls.

1. A method for a target from a biological sample, the method comprising:
a. Mixing the biological sample with paramagnetic particles (PMPs) to generate a composition comprising one or more target-PMP complexes within a pipette tip;
b. Generating a liquid/air interface proximal to a bottom opening of the pipette tip; and
c. Applying a magnetic force to the composition to draw target-PMP complexes through the liquid/air interface and into a sample collection device.

2. A method for separating a target from a biological sample, the method comprising performing the following steps, in order:
a. Mixing the biological sample with paramagnetic particles (PMPs) to generate a composition comprising one or more target-PMP complexes;
b. Aspirating the composition into a pipette tip through a bottom opening of the pipette tip and generating a liquid/air interface proximal to the bottom opening of the pipette tip, or aspirating the composition into a pipette tip through a side opening of the pipette tip while a bottom opening of the pipette tip is in conformational contact with a surface such that liquid is unable to enter the pipette tip through the bottom opening and generating a liquid/air interface proximal to the bottom opening of the pipette tip; and
Applying a magnetic force the composition to draw target-PMP complexes through the liquid/air interface and into a sample collection device.

3. The method of claim 2, wherein generating the liquid/air interface proximal to the bottom opening of the pipette tip comprises further aspirating the composition within the pipette tip while the bottom opening of the pipette tip is exposed to air.

4. (canceled)

5. (canceled)

6. (canceled)

7. (canceled)

8. The method of claim 2, wherein the method comprises applying a magnetic force to the composition to generate a concentration of target-PMP complexes proximal to the liquid/air interface prior to drawing the target-PMP complexes through the liquid/air interface and into the sample collection device.

9. The method of claim 2, wherein the biological sample is a nasopharyngeal sample, an oropharyngeal sample, an oral swab sample, an oral sponge sample, a nasal swab sample, a mid-turbinate sample, or a saliva sample.

10. (canceled)

11. (canceled)

12. The method of claim 2, wherein the sample collection device comprises a wash buffer.

13. The method of claim 12, further comprising:
a. removing the wash buffer from the sample collection device; and
b. adding reagents for detection of the target to the sample collection device after removal of the wash buffer.

14. The method of claim 2, wherein the sample collection device comprises reagents for detection of the target.

15. The method of claim 2, wherein the sample collection device comprises a multi-well plate.

16. The method of claim 2, where in the sample collection device further comprises a layer of mineral oil, wherein the layer of mineral oil floats above the reagents for detection of the target and/or the wash buffer.

17. The method of claim 2, wherein the biological sample is obtained from a subject suspected of having an infection.

18. The method of claim 17, wherein the subject is suspected of having a viral infection, a viral upper respiratory infection, or an infection due to SARS-CoV2, coronavirus, rhinovirus, influenza, respiratory syncytial virus adenovirus, parainfluenza, human immunodeficiency virus, human papillomavirus, rotavirus, hepatitis C virus, zika virus, Ebola virus, tuberculosis, Borrelia burgdorferi, staphylococcus, aspergillus, or Streptococcus pyrogenes.

19. (canceled)

20. (canceled)

21. The method of claim 2, wherein the target comprises a material selected from the group consisting of viral nucleic acids, bacterial nucleic acids, proteins, glycopeptides, antibodies, cells, DNA sequences, RNA sequences, lipids, and carbohydrates.

22. The method of claim 2, wherein the method is automated.

23. (canceled)

24. (canceled)

25. The method of claim 2, further comprising detecting the target within the sample collection device.

26. A system for automated isolation of a target from a plurality of biological samples, the system comprising the following components:
a. A plurality of pipette tips;
b. A multichannel pipette;
c. An apparatus for operating the multichannel pipette;
d. A preconcentration magnet;
e. A sample collection device;
f. A sample collection magnet; and
g. A computer processor configured to operate said multichannel pipette so as to collect a liquid sample in said plurality of pipette tips when attached to said multichannel pipette and to generate an air gap at an open end of said pipette tips following or during collection of said liquid sample.

27. The system of claim 26, further comprising a moveable surface, wherein one or more of the components are housed on the moveable surface and the moveable surface changes orientation and/or moves in the x-y plane and/or in the vertical z-direction.

28. (canceled)

29. The system of claim 26, further comprising a plurality of biological samples, wherein each of the plurality of biological samples occupies a separate well in a multi-well plate and, optionally, wherein the plurality of pipette tips and/or each well in the multi-well plate contain a lyophilized or liquid formulation of paramagnetic particles (PMPs).

30. (canceled)

31. The system of claim 26, further comprising a mixing container for combining the biological sample with paramagnetic particles (PMPs).

32. (canceled)

33. The system of claim 26, wherein the sample collection device comprises a multi-well plate, the sample collection magnet is positioned below the sample collection device, the apparatus for operating the multichannel pipette is configured to induce movement of the multichannel pipette, and/or the apparatus for operating the multichannel pipette is configured to aspirate and/or inject liquid which pipette tips are attached to the multichannel pipette.

34. (canceled)

35. (canceled)

36. (canceled)

37. The system of claim 26, wherein the system comprises a moveable surface, and wherein the moveable surface is controlled by a computer.

38. The method of claim 2, wherein the biological sample is mixed with PMPs contained within the pipette tip to generate the composition comprising one or more target-PMP complexes within the pipette tip.
